(19) United States
(12) Patent Application Publication
(10) Pub. No.: US 2022/0203015 A1
(43) Pub. Date: Jun. 30, 2022

(54) DATA RECORDER IN WOUND THERAPY SYSTEMS

(71) Applicant: T.J.Smith and Nephew,Limited, Hull (GB)

(72) Inventors: Edward Yerbury Hartwell, Hull (GB); Felix Clarence Quintanar, Hull (GB)

(21) Appl. No.: 17/601,020

(22) PCT Filed: Mar. 19, 2020
(86) PCT No.: PCT/EP2020/057641

§ 371 Date: Oct. 1, 2021

(30) Foreign Application Priority Data
Apr. 3, 2019 (GB) 1904686.1

(51) Int. Cl. A61M 1/00

(52) U.S. Cl. CPC A61M 1/90; A61M 2205/15; A61M 2205/3344; A61M 2205/17

(57) ABSTRACT

Embodiments of negative pressure wound therapy systems and methods for operating the systems are disclosed. In one embodiment, an apparatus includes a first housing, a pressure source, a first controller, a second housing, a sensor, and a second controller. The pressure source can be supported by the first housing and couple via a fluid flow path to a wound dressing positioned on a wound and provide negative pressure to the wound. The first controller configured to operate the pressure source. The second housing can be supported by the first housing. The sensor can be within the second housing and generate sensor data usable to troubleshoot an error condition associated with performance of pressure therapy with the first controller. The second controller can be within the second housing and the sensor data to a memory device.

### CROSS-REFERENCE TO RELATED APPLICATIONS

This application claims priority to U.K. Provisional Application No. 1904686.1 filed on Apr. 3, 2019; the disclosure of which is hereby incorporated by reference in its entirety.

### BACKGROUND

### Field

Embodiments of the present disclosure relate to methods and apparatuses for dressing and treating a wound with negative or reduced pressure therapy or topical negative pressure (TNP) therapy. In particular, but without limitation, embodiments disclosed herein relate to negative pressure therapy devices, methods for controlling the operation of TNP systems, and methods of using TNP systems.

### Description of the Related Art

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds and abdominal wounds or the like.

TNP therapy assists in the closure and healing of wounds by reducing tissue oedema, encouraging blood flow, stimulating the formation of granulation tissue, removing excess exudates and may reduce bacterial load and, thus, infection to the wound. Furthermore, TNP therapy permits less outside disturbance of the wound and promotes more rapid healing.

### SUMMARY

In some aspects, an apparatus for applying pressure to a wound is disclosed. The apparatus can include a first housing, a pressure source, a first controller, a second housing, a sensor, and a second controller. The pressure source can be supported by the first housing and couple via a fluid flow path to a wound dressing positioned on a wound and provide negative pressure to the wound. The first controller can operate the pressure source. The second housing can be supported by the first housing. The sensor can be within the second housing and generate sensor data usable to troubleshoot an error condition associated with performance of pressure therapy with the first controller. The second controller can be within the second housing and store the sensor data to a memory device. The second controller may be different from the first controller.

The apparatus of the preceding paragraph can include one or more of the following features: The second controller can receive device data from the first controller and store the device data to the memory device. The device data can include device events or parameter measurements that are associated with operation of the first controller or the pressure source. The second controller can add an entry to a log indicating an occurrence of the error condition. The second controller can determine a frequency of occurrence of the error condition from the log. The second housing can be fireproof or shockproof. The memory device can be non-wipeable or non-resettable. The second controller can output the sensor data from the memory device to another device. The sensor can be a temperature sensor or a motion sensor. The first controller and the second controller can be powered by different power sources. The second controller can determine a cause of the error condition from the sensor data and output an indication of the cause for presentation on a display. The second controller can determine and output a user instruction indicating how to remedy the error condition. The apparatus can further include a display supported by the second housing, and the display can include electronic ink or one or more light sources. The second controller may not be configured to operate the pressure source or communicate with the first controller. The error condition can include a blockage in the fluid flow path or a low pressure level at the wound. The error condition can prevent performance of the pressure therapy with the first controller.

In some aspects, a method can include: generating, with a sensor, sensor data usable to troubleshoot an error condition associated with operation of a wound therapy device, the sensor being positioned in a recorder housing that is positioned in a device housing of the wound therapy device; storing the sensor data to a memory device positioned in the recorder housing; and outputting the sensor data from the memory device to an electronic device.

The method of the preceding paragraph can include one or more of the following features: The method can include removing the recorder housing from the device housing. The sensor can be a motion sensor, and the sensor data can be motion data. The sensor can be a temperature sensor, and the sensor data can be temperature data. The method can include determining, with a processor, a cause of the error condition from the sensor data.

### BRIEF DESCRIPTION OF THE DRAWINGS

Embodiments of the present disclosure will now be described hereinafter, by way of example only, with reference to the accompanying drawings in which:

FIG. 1 illustrates a negative pressure wound therapy system.

FIGS. 2A, 2B, and 2C illustrate a TNP apparatus.

FIG. 3 illustrates components of a negative pressure wound therapy system.

FIGS. 4 and 5 illustrate troubleshooting processes performable in a negative pressure wound therapy system.

### DETAILED DESCRIPTION

The present disclosure relates to methods and apparatuses for dressing and treating a wound with reduced pressure therapy or topical negative pressure (TNP) therapy. In particular, but without limitation, embodiments of this disclosure relate to negative pressure therapy apparatuses, methods for controlling the operation of TNP systems, and methods of using TNP systems. The methods and apparatuses can incorporate or implement any combination of the features described below. Moreover, the features of this disclosure can be incorporated or implemented in other wound therapy apparatuses, such as positive pressure therapy devices, or other medical apparatuses usable for treating a patient.

TNP therapy can assist in the closure and healing of many forms of “hard to heal” wounds by reducing tissue oedema, encouraging blood flow and granular tissue formation, or removing excess exudate and can reduce bacterial load (and thus infection risk). In addition, TNP therapy may allow for less disturbance of a wound leading to more rapid healing. TNP systems can also assist in the healing of surgically closed wounds by removing fluid or help to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels that are below atmospheric pressure, which typically corresponds to 760 mmHg (or 1 atm, 29.93 inHg, 101.325 kPa, 14.696 psi, etc.). Accordingly, a negative pressure value of −X mmHg reflects pressure that is X mmHg below atmospheric pressure, such as a pressure of (760−X) mmHg. In addition, negative pressure that is “less” or “smaller” than −X mmHg corresponds to pressure that is closer to atmospheric pressure (e.g., −40 mmHg is less than −60 mmHg). Negative pressure that is “more” or “greater” than −X mmHg corresponds to pressure that is further from atmospheric pressure (e.g., −80 mmHg is more than −60 mmHg).

### Overview

A data recorder can be incorporated as part of or within a TNP apparatus. The data recorder can include a recorder housing that contains or supports a recorder controller, a recorder memory device, and one or more recorder sensors. The recorder housing can be robust (for example, fireproof or shockproof) so that the recorder housing may protect components of the data recorder in the event of a damaging event to the TNP apparatus, such as a fire, shock, or exposure to high temperatures. The recorder housing can be contained in or supported by a device housing of the TNP apparatus. The recorder controller can be separate from and may not communicate with a device controller of the TNP apparatus that may control other functions of the TNP apparatus, such as operation of a pressure source. Components of the data recorder may not communicate with components of the TNP apparatus other than those components that are part of the data recorder. The recorder memory device can be non-wipeable or non-resettable to help protect the sensor data collected by the data recorder from being changed, degraded, or destroyed. The data recorder or its components or functionality can be inaccessible to a patient or a clinician. The data recorder or its components or functionality may instead be accessible to a technician, which can be an individual tasked with analyzing or troubleshooting the TNP apparatus.

The data recorder can include a power supply that is separate from a power supply used for powering other components of the TNP apparatus, such as a pressure source or a controller for operating the pressure source. The data recorder may accordingly continue to operate despite one or more other components of the TNP apparatus no longer operating as expected or continuing to operate.

The data recorder can collect sensor data, such as by its one or more sensors, usable for troubleshooting an error condition that impacts a functionality of the TNP apparatus, such as a failure or malfunction of the TNP apparatus or a misuse or non-compliant use of the TNP apparatus. The error condition may be caused by improper handling of the TNP apparatus by a user, exposure to extreme environmental conditions like a high temperature, or a malfunction of a component of the TNP apparatus like a short in a power source. In one example, the data recorder can collection motion data with its one or more sensors that is usable to determine why a leak or a blockage may have occurred (which may be due to an inversion of the TNP apparatus, a sudden impact to the TNP apparatus, or an excess vibration of the TNP apparatus that may have caused a filter between a canister and a pump assembly of the TNP apparatus to become occluded). In another example, the data recorder can collect temperature data indicative of an ambient temperature around the TNP apparatus, and the temperature data may be usable to determine that the TNP apparatus was exposed to a temperature that exceeded a permissible level for a component of the TNP apparatus, such as a power source, so that the TNP apparatus may fail to operate properly (for instance, because the power source may fail to charge or provide power to a pressure source, which can cause the TNP apparatus to at least temporarily be unable to perform wound therapy).

The data recorder can process the sensor data to identify misuse or non-compliant use of the TNP apparatus or may provide guidance, such as training or troubleshooting advice, to assist a user in addressing an error condition that impacts the functionality of the TNP apparatus. The data recorder may include a display (for instance, an electronic ink display) or one or more light sources (for instance, light emitting diodes) via which the data recorder may present information to a user. The data recorder may store the identified misuse or non-compliant use or guidance to the recorder memory device.

The sensor data collected by the data recorder may be particularly helpful for troubleshooting a temporary or transient error because the sensor data may provide useful information about the TNP apparatus prior to the error, during the error, and subsequent to the error. Without the data recorder, the TNP apparatus may not, in certain instances, retain sufficient information for understanding the conditions that may have existed around the time of the error, and the TNP apparatus instead may just retain information about the occurrence of the error that may prove insufficient for successful troubleshooting of the error.

The data recorder can include security and self-testing features. For example, the recorder housing of the data recorder may be tamper-proof to prevent tampering (such as by being locked or password-protected), tamper-evident to indicate when tampering has occurred (such as by recording a tamper event to the recorder memory device, reporting a tamper event by sending a message to a remote device via a computer network, or incorporating a mechanical mechanism which irreversibly or reversibly changes responsive to a tamper event), or self-protecting to defend against tampering (such as configured to wipe its recorder memory device or ruin a functionality or hardware of the data recorder responsive to a potential or actual tamper event that is detected). As another example, the data recorder may include one or more cybersecurity protections to prevent unauthorized remote access to or communication of the sensor data or other information determined by the data recorder. As yet another example, the recorder memory device or other hardware of the data recorder can be self-testing to run diagnostics to confirm correct functioning, as well as to potentially perform error correcting.

### Wound Therapy Systems

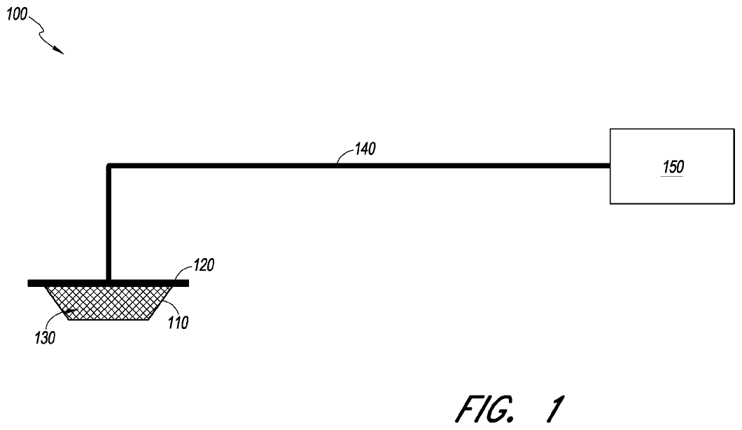

FIG. 1 illustrates a negative or reduced pressure wound treatment (or TNP) system 100 comprising a wound filler 130 placed inside a wound cavity 110, the wound cavity sealed by a wound cover 120. The wound filler 130 in combination with the wound cover 120 can be referred to as wound dressing. A single or multi lumen tube or conduit 140 is connected to the wound cover 120 with a TNP apparatus 150 configured to supply reduced pressure. The wound cover 120 can be in fluidic communication with the wound cavity 110. The TNP apparatus can be a canisterless TNP apparatus (meaning that exudate is collected in the wound dressing or is transferred via conduit 140 for collection to another location) or configured to include or support a canister. Additionally, the TNP apparatus can be mounted to or supported by the dressing, or adjacent to the dressing.

The wound filler 130 can be any suitable type, such as hydrophilic or hydrophobic foam, gauze, inflatable bag, and so on. The wound filler 130 can be conformable to the wound cavity 110 such that it substantially fills the cavity. The wound cover 120 can provide a substantially fluid impermeable seal over the wound cavity 110. The wound cover 120 can have a top side and a bottom side, and the bottom side adhesively (or in any other suitable manner) seals with wound cavity 110. The conduit 140 or lumen or any other conduit or lumen disclosed herein can be formed from polyurethane, PVC, nylon, polyethylene, silicone, or any other suitable material.

The wound cover 120 can have a port (not shown) configured to receive an end of the conduit 140. For example, the port can be Renasys Soft Port available from Smith & Nephew. Additionally or alternatively, the conduit 140 can otherwise pass through or under the wound cover 120 to supply reduced pressure to the wound cavity 110 so as to maintain a desired level of reduced pressure in the wound cavity. The conduit 140 can be any suitable article configured to provide at least a substantially sealed fluid flow pathway between the TNP apparatus 150 and the wound cover 120, so as to supply the reduced pressure provided by the TNP apparatus 150 to wound cavity 110.

The wound cover 120 and the wound filler 130 can be provided as a single article or an integrated single unit, or no wound filler may be provided and the wound cover 120 by itself may be considered the wound dressing. The wound dressing may then be connected, via the conduit 140, to a source of negative pressure, such as the TNP apparatus 150. The TNP apparatus 150 can be miniaturized and portable, although larger conventional pumps such can also be used.

The wound cover 120 can be located over a wound site to be treated. The wound cover 120 can form a substantially sealed cavity or enclosure over the wound site. The wound cover 120 can have a film having a high water vapor permeability to enable the evaporation of surplus fluid, and can have a superabsorbing material contained therein to safely absorb wound exudate. It will be appreciated that throughout this specification reference is made to a wound. In this sense it is to be understood that the term wound is to be broadly construed and encompasses open and closed wounds in which skin is torn, cut or punctured or where trauma causes a contusion, or any other surficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, acute wounds, chronic wounds, surgical incisions and other incisions, subacute and dehisced wounds, traumatic wounds, flaps and skin grafts, lacerations, abrasions, contusions, burns, diabetic ulcers, pressure ulcers, stoma, surgical wounds, trauma and venous ulcers or the like. The components of the TNP system described herein can be particularly suited for incisional wounds that exude a small amount of wound exudate.

Some embodiments of the system are designed to operate without the use of an exudate canister. Some embodiments can be configured to support an exudate canister. Configuring the TNP apparatus 150 and conduit 140 so that the conduit 140 can be quickly and easily removed from the TNP apparatus 150 can facilitate or improve the process of dressing or pump changes, if necessary. The TNP apparatus 150 can be configured to have any suitable connection between the tubing and the pump.

The TNP apparatus 150 can be configured to deliver negative pressure of approximately −80 mmHg, or between about −20 mmHg and −200 mmHg in some implementations. Note that these pressures are relative to normal ambient atmospheric pressure thus, −200 mmHg would be about 560 mmHg in practical terms. The pressure range can be between about −40 mmHg and −150 mmHg. Alternatively a pressure range of up to −75 mmHg, up to −80 mmHg or over −80 mmHg can be used. Also a pressure range of below −75 mmHg can be used. Alternatively a pressure range of over approximately −100 mmHg, or even −150 mmHg, can be supplied by the TNP apparatus 150.

In operation, the wound filler 130 is inserted into the wound cavity 110 and wound cover 120 is placed so as to seal the wound cavity 110. The TNP apparatus 150 provides a source of a negative pressure to the wound cover 120, which is transmitted to the wound cavity 110 via the wound filler 130. Fluid (e.g., wound exudate) is drawn through the conduit 140, and can be stored in a canister. Fluid can be absorbed by the wound filler 130 or one or more absorbent layers (not shown).

Wound dressings that may be utilized with the TNP apparatus and other embodiments of the present application include Renasys-F, Renasys-G, Renasys AB, and Pico Dressings available from Smith & Nephew. Further description of such wound dressings and other components of a negative pressure wound therapy system that may be used with the TNP apparatus and other embodiments of the present application are found in U.S. Patent Publication Nos. 2011/0213287, 2011/0282309, 2012/0116334, 2012/0136325, and 2013/0110058, which are incorporated by reference in their entirety. In other embodiments, other suitable wound dressings can be utilized.

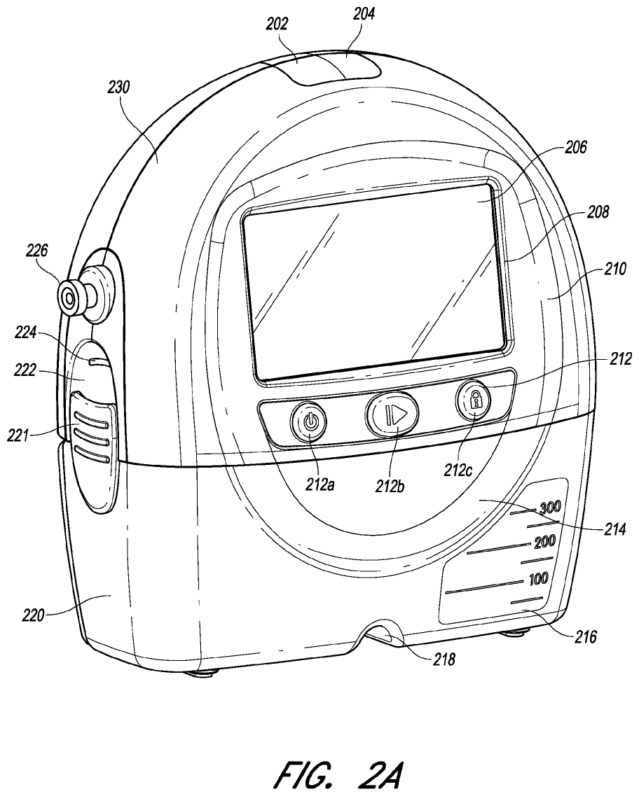

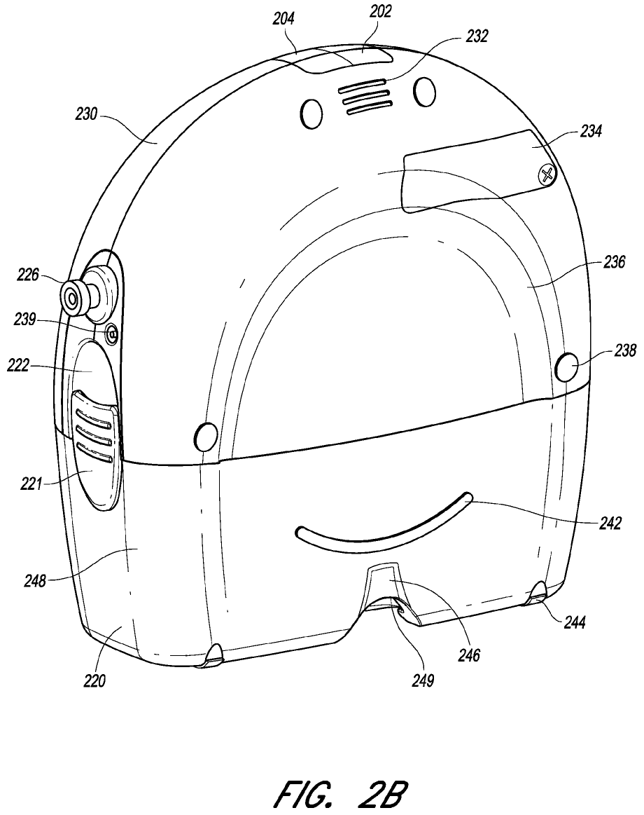

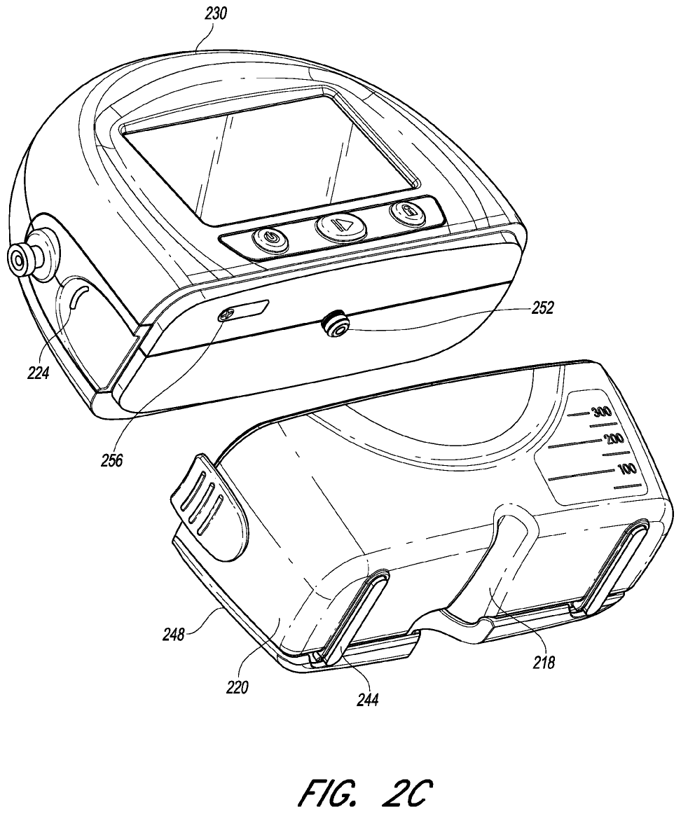

FIG. 2A illustrates a front view of a pump assembly 230 and canister 220. As is illustrated, the pump assembly 230 and the canister 220 are connected, thereby forming a TNP apparatus. The pump assembly 230 can be similar to or the same as the TNP apparatus 150.

The pump assembly 230 includes one or more indicators, such as visual indicator 202 configured to indicate alarms and visual indicator 204 configured to indicate status of the TNP system. The visual indicators 202 and 204 can be configured to alert a user, such as patient or medical care provider, to a variety of operating or failure conditions of the system, including alerting the user to normal or proper operating conditions, pump failure, power supplied to the pump or power failure, detection of a leak within the wound cover or flow pathway, suction blockage, or any other similar or suitable conditions or combinations thereof. The pump assembly 230 can comprise additional indicators. The pump assembly can use a single indicator or multiple indicators. Any suitable indicator can be used such as visual, audio, tactile indicator, and so on. The visual indicator 202 can be configured to signal alarm conditions, such as canister full, power low, conduit 140 disconnected, seal broken in the wound cover 120, and so on. The visual indicator 202 can be configured to display red flashing light to draw user's attention. The visual indicator 204 can be configured to signal status of the TNP system, such as therapy delivery is ok, leak detected, and so on. The visual indicator 204 can be configured to display one or more different colors of light, such as green, yellow, etc. For example, green light can be emitted when the TNP system is operating properly and yellow light can be emitted to indicate a warning.

The pump assembly 230 includes a display or screen 206 mounted in a recess 208 formed in a case of the pump assembly. The display 206 can be a touch screen display. The display 206 can support playback of audiovisual (AV) content, such as instructional videos. As explained below, the display 206 can be configured to render a number of screens or graphical user interfaces (GUIs) for configuring, controlling, and monitoring the operation of the TNP system. The pump assembly 230 comprises a gripping portion 210 formed in the case of the pump assembly. The gripping portion 210 can be configured to assist the user to hold the pump assembly 230, such as during removal of the canister 220. The canister 220 can be replaced with another canister, such as when the canister 220 has been filled with fluid.

The pump assembly 230 includes one or more keys or buttons configured to allow the user to operate and monitor the operation of the TNP system. As is illustrated, there buttons 212a, 212b, and 212c (collectively referred to as buttons 212) are included. Button 212a can be configured as a power button to turn on/off the pump assembly 230. Button 212b can be configured as a play/pause button for the delivery of negative pressure therapy. For example, pressing the button 212b can cause therapy to start, and pressing the button 212b afterward can cause therapy to pause or end. Button 212c can be configured to lock the display 206 or the buttons 212. For instance, button 212c can be pressed so that the user does not unintentionally alter the delivery of the therapy. Button 212c can be depressed to unlock the controls. In other embodiments, additional buttons can be used or one or more of the illustrated buttons 212a, 212b, or 212c can be omitted. Multiple key presses or sequences of key presses can be used to operate the pump assembly 230.

The pump assembly 230 includes one or more latch recesses 222 formed in the cover. In the illustrated embodiment, two latch recesses 222 can be formed on the sides of the pump assembly 230. The latch recesses 222 can be configured to allow attachment and detachment of the canister 220 using one or more latches 221. The pump assembly 230 comprises an air outlet 224 for allowing air removed from the wound cavity 110 to escape. Air entering the pump assembly can be passed through one or more suitable filters, such as antibacterial filters. This can maintain reusability of the pump assembly. The pump assembly 230 includes one or more strap mounts 226 for connecting a carry strap to the pump assembly 230 or for attaching a cradle. In the illustrated embodiment, two strap mounts 226 can be formed on the sides of the pump assembly 230. In some embodiments, various of these features are omitted or various additional features are added to the pump assembly 230.

The canister 220 is configured to hold fluid (e.g., exudate) removed from the wound cavity 110. The canister 220 includes the one or more latches 221 for attaching the canister to the pump assembly 230. In the illustrated embodiment, the canister 220 comprises two latches 221 on the sides of the canister. The exterior of the canister 220 can formed from frosted plastic so that the canister is substantially opaque and the contents of the canister and substantially hidden from plain view. The canister 220 comprises a gripping portion 214 formed in a case of the canister. The gripping portion 214 can be configured to allow the user to hold the pump assembly 230, such as during removal of the canister from the pump assembly 230. The canister 220 includes a substantially transparent window 216, which can also include graduations of volume. For example, the illustrated 300 mL canister 220 includes graduations of 50 mL, 100 mL, 150 mL, 200 mL, 250 mL, and 300 mL. Other embodiments of the canister can hold different volume of fluid and can include different graduation scale. For example, the canister can be an 800 mL canister. The canister 220 comprises a tubing channel 218 for connecting to the conduit 140. In some embodiments, various of these features, such as the gripping portion 214, are omitted or various additional features are added to the canister 220. Any of the disclosed canisters may include or may omit a solidifier.

FIG. 2B illustrates a rear view of the pump assembly 230 and canister 220. The pump assembly 230 comprises a speaker port 232 for producing sound. The pump assembly 230 includes a filter access door 234 with a screw for removing the filter access door 234, accessing, and replacing one or more filters, such as antibacterial or odor filters. The pump assembly 230 comprises a gripping portion 236 formed in the case of the pump assembly. The gripping portion 236 can be configured to allow the user to hold the pump assembly 230, such as during removal of the canister 220. The pump assembly 230 includes one or more covers 238 configured to as screw covers or feet or protectors for placing the pump assembly 230 on a surface. The pump assembly 230 comprises a power jack 239 for charging and recharging an internal battery of the pump assembly. The power jack 239 can be a direct current (DC) jack. The pump assembly can comprise a disposable power source, such as batteries, so that no power jack is needed.

The canister 220 includes one or more feet 244 for placing the canister on a surface. The canister 220 comprises a tube mount relief 246 configured to allow one or more tubes to exit to the front of the device. The canister 220 includes a stand or kickstand 248 for supporting the canister when it is placed on a surface. The kickstand 248 can pivot between an opened and closed position. The kickstand 248 includes a gripping portion 242 formed in the kickstand. The gripping portion 242 can be configured to allow the user to place the kickstand 248 in the closed position. The kickstand 248 comprises a hole 249 to allow the user to place the kickstand in the open position.

FIG. 2C illustrates a view of the pump assembly 230 separated from the canister 220. The pump assembly 230 includes a vacuum attachment, connector, or inlet 252 through which a vacuum pump communicates negative pressure to the canister 220. The pump assembly aspirates fluid, such as gas, from the wound via the inlet 252. The pump assembly 230 comprises a USB access door 256 configured to allow access to one or more USB ports. The USB access door is omitted and USB ports are accessed through the filter access door 234. The pump assembly 230 can include additional access doors configured to allow access to additional serial, parallel, or hybrid data transfer interfaces, such as SD, Compact Disc (CD), DVD, FireWire, Thunderbolt, PCI Express, and the like. In other embodiments, one or more of these additional ports are accessed through the filter access door 234.

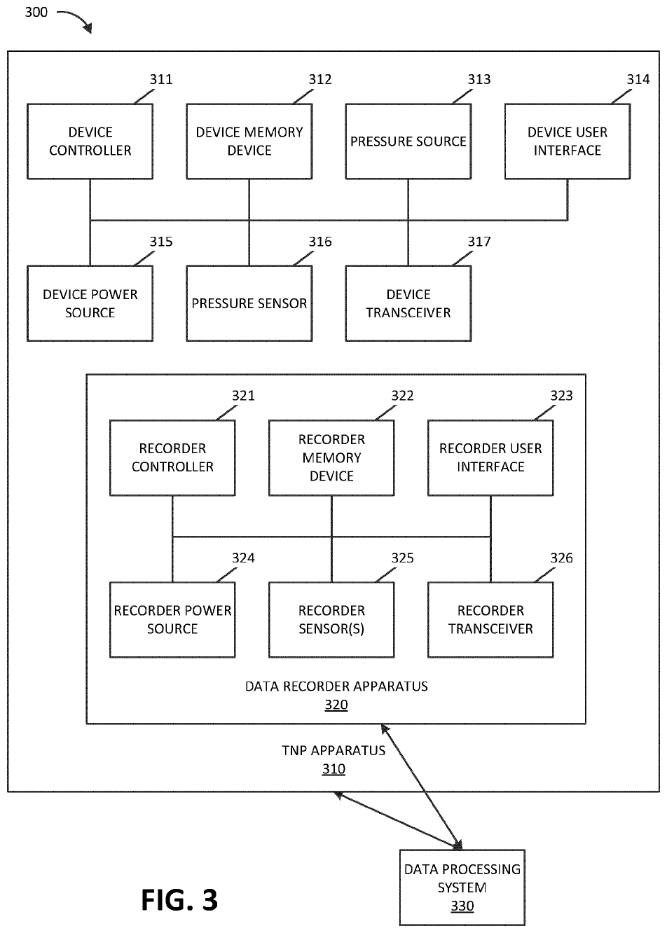

FIG. 3 illustrates components of a negative pressure wound therapy system 300 that includes a TNP apparatus 310 and a data processing system 330. The TNP apparatus 310 can be used to treat a wound using a wound dressing in fluidic communication with the TNP apparatus 310 via a fluid flow path.

As illustrated by FIG. 3, the TNP apparatus 310 can include a device controller 311, a device memory device 312, a pressure source 313, a device user interface 314, a device power source 315, a pressure sensor 316, and a device transceiver 317 that may communicate, such as electrically, with one another and can be supported by or contained in a device housing of the TNP apparatus 310. The device power source 315 can provide power to one or more components of the TNP apparatus 310. The TNP apparatus 310 can operate at pressure levels and using control approaches as described herein or similar to those described in U.S. Patent Publication Nos. 2016/0136339 and 2016/0184496, which are incorporated by reference in their entirety. The TNP apparatus 310 can be similar to or the same as the TNP apparatus 150.

The device controller 311 can control operations of one or more other components of the TNP apparatus 310 according at least to instructions stored in the device memory device 312. The device controller 311 can, for instance, control operations of and supply of negative pressure by the pressure source 313. The pressure source 313 can include a pump, such as a rotary diaphragm pump or other diaphragm pump, a piezoelectric pump, a peristaltic pump, a piston pump, a rotary vane pump, a liquid ring pump, a scroll pump, a diaphragm pump operated by a piezoelectric transducer, or any other suitable pump or micropump or any combinations of the foregoing.

The device user interface 314 can include one or more elements that receive user inputs or provide user outputs to a patient or caregiver. The one or more elements that receive user inputs can include buttons, switches, dials, touch screens, or the like. The device user interface 314 can, for example, be used to generate and display a report or other information reflecting data from therapy use, data from non-compliant use, or a comparison of data from therapy use versus non-compliant use. As another example, the device user interface 314 may receive a user input that provides a patient reference number or another unique identifier, and the TNP apparatus 310 may then be activated for use by the patient and data collected and associated with the patient reference number for usage monitoring for a particular patient. The device user interface 314 can provide an alert to the user. For example, the device user interface 314 can include a screen that may visibly present the alert or a speaker that may audibly present the alert.

The pressure sensor 316 can be used to monitor pressure underneath a wound dressing, such as pressure in a fluid flow path connecting the pressure source 313 and the wound dressing, pressure at the wound dressing, or pressure at or in the pressure source 313. As the pressure source 313 provides negative pressure, the pressure source 313 may generate pressure pulses that are propagated through the fluid flow path and detected by the pressure sensor 316. These pressure pulses may show as a change or bounce in the magnitude or frequency of a signal from the pressure sensor 316.

The device controller 311 can analyze a signal output by the pressure sensor 316 to determine pressure in the fluid flow path. The device controller 311 may examine the signal using one or more approaches including time domain or frequency domain calculations, such as with a digital signal processor.

The detection of pressure by the pressure sensor 316 can, in some implementations, be enhanced by changing one or more settings of the pressure source 313, such as increasing or decreasing vacuum level delivered by the pressure source 313, stopping the pressure source 313, changing an operating speed of the pressure source 313, changing a cadence of the pressure source 313, combinations of the same, or the like. The device controller 311 can, for example, automatically manage adjustment of the one or more settings.

The pressure sensor 316 can be used in combination with another pressure sensor so that the at least two pressure sensors that are positioned in or fluidically connected to the fluid flow path to permit differential measurement of the pressure. For example, a first pressure sensor can be positioned upstream of the wound (such as at or near the inlet of the pressure source 313) and a second pressure sensor can be positioned to detect pressure at or near the wound or at or near a canister. This configuration can be accomplished by incorporating, in addition to one or more lumens forming a first fluid flow path connecting the pressure source 313 to the wound, a second fluid flow path that includes one or more lumens connecting the TNP apparatus 310 to the wound and through which the second pressure sensor can monitor pressure at or near the wound or at or near a canister. The first and second fluid flow paths can be fluidically isolated from each other. When the at least two pressure sensors are used, the rate of change of pressure (for example, in peak-to-peak pressure or maximum pressure) in the first and second fluid flow paths can be determined and the difference in pressure detected between the first and second pressure sensors can be determined. These values can be used separately or together to detect various operational conditions, such as leaks, blockages, canister full, presence of blood in the first fluid flow path or the second fluid flow path, etc. In some implementations, multiple redundant pressure sensors can be provided to protect against failure of individual pressure sensors.

The device transceiver 317 can be used to communicate with the data processing system 330. The device transceiver 317 can, for example, transmit device usage data like alarms, measured pressure, or changes to a therapy program administered by the TNP apparatus 310 to the data processing system 330, such as via wired or wireless communications (for example, via a communication network like a cellular communications network). The device memory device 312 can be used to store the device usage data that may be transmitted by the device transceiver 317.

The TNP apparatus 310 can include a data recorder apparatus 320 that may be supported by or contained in the device housing of the TNP apparatus 310. The data recorder apparatus 320 can collect data usable for troubleshooting an error condition at the TNP apparatus 310, such as a failure or malfunction of the TNP apparatus 310 due to adverse environmental conditions or misuse or non-compliant use of the TNP apparatus 310. The collected data may be particularly helpful for troubleshooting a temporary or transient error. Moreover, the data recorder apparatus 320 may display information related to or troubleshoot the error condition.

As further illustrated in FIG. 3, the data recorder apparatus 320 can include a recorder controller 321, a recorder memory device 322, a recorder user interface 323, a recorder power source 324, one or more recorder sensors 325, and a recorder transceiver 326. One or more components of the data recorder apparatus 320 can be supported by or contained in a recorder housing of the data recorder apparatus 320. The recorder housing can be fireproof or shockproof, which may protect the data recorder apparatus 320 and its components from fire or shocks to the TNP apparatus 310 that can cause damage to or problems for operation of the data recorder apparatus 320. The recorder housing can include a polymer that physically protects components of the data recorder apparatus 320, such as from mechanical disturbances or exposure to high temperatures. The recorder housing can have a length or a width ranging from 1 cm to 20 cm, 2 cm to 15 cm, 2 cm to 10 cm, or 2 cm to 5 cm. The length or width can be less the 20 cm, 15 cm, 10 cm, 9 cm, 8 cm, 7 cm, 6 cm, 5 cm, 4 cm, 3 cm, 2 cm, or 1 cm. The recorder housing can be shaped as a cuboid, a triangular prism, or another shape. The recorder housing can be separable from the device housing so that the data recorder apparatus 320 can be interrogated separately from the TNP apparatus 310.

The recorder controller 321 can control operations of one or more other components of the data recorder apparatus 320 according at least to instructions stored in the recorder memory device 322. The recorder controller 321 can, for instance, monitor with the one or more recorder sensors 325 one or more parameters relevant a functionality of the TNP apparatus 310 (such as performance of wound therapy, operation of the device power source 315, or operation of the pressure source 313) and store corresponding sensor data to the recorder memory device 322. As another example, the recorder controller 321 can receive device data (such as (i) device events like alarms or device responses to alarms or (ii) parameter measurements or vital sign indications that are associated with operation or functionality of the device controller 311, the device memory device 312, the pressure source 313, the device user interface 314, the device power source 315, the pressure sensor 316, or the device transceiver 317) from the device transceiver 317 via the recorder transceiver 326 and store the received device data to the recorder memory device 322. The recorder controller 321 can detect an error condition from the sensor data or the device data, add an entry to a log indicating an occurrence of the error condition, determine a frequency of occurrence of the error condition from the log, determine a cause of the error condition from the sensor data or the device data, or output an indication of the cause or a user instruction indicating how to remedy the error condition for presentation on a display. The recorder controller 321 may not operate the pressure source 313 and can be separate from the device controller 311.

The recorder memory device 322 can be non-wipeable or non-resettable so that the sensor data stored to the recorder memory device 322 may be robustly or securely stored to the recorder memory device 322 and not be erased by a user or adverse environmental conditions. In some implementations, the recorder memory device 322 can be wipeable and may discard any data, such as the sensor data or the device data, once the data has been communicated to another device, such as the data processing system 330.

The recorder user interface 323 can include one or more elements that receive user inputs or provide outputs to a user that desires to troubleshoot the TNP apparatus 310. The one or more elements that receive user inputs can include buttons, switches, dials, touch screens, or the like. The recorder user interface 323 can, for example, be used to display, such with an electronic ink display or one or more light sources, information indicative of an error condition detected by the data recorder apparatus 320 or how to troubleshoot the error condition (such as to replace a filter of the TNP apparatus 310) or not to repeat the cause of the error condition (such as not indicating not to rotate or vibrate the housing of the TNP apparatus 310).

The recorder power source 324 can provide power to one or more components of the data recorder apparatus 320 and may be different from or the same as the device power source 315. The recorder power source 324 can include a battery or a super capacitor. The recorder power source 324 can receive power from the device power source 315, such as to recharge, or may operate independently from and not communicate with or receive power from the device power source 315. If the recorder power source 324 is different from and operates independently from the device power source 315, the data recorder apparatus 320 can be powered separately from other components of the TNP apparatus 310 so that the data recorder apparatus 320 may continue to operate despite problems with or loss of power at the device power source 315.

The one or more recorder sensors 325 can be usable to measure a parameter associated with a safety or successful functioning of the TNP apparatus 310. For example, the one or more recorder sensors 325 can include one or more motion sensors (for example, an accelerometer, gyroscope, inertial measurement unit, or orientation detector) or temperature sensors (for example, a thermistor, resistance temperature detector, thermocouple, or semiconductor-based sensor), among other possible types of sensors. The one or more motion sensors may detect one or more parameters, such as acceleration in x, y, z direction or an angle between the orientation of the TNP apparatus 310 and a direction of gravity. The one or more temperature sensors can detect a temperature at one or more positions in or around the data recorder apparatus 320, which may be indicative of a temperature at one or more positions in or around the TNP apparatus 310.

The output from the one or more recorder sensors 325 can provide information about various conditions of or situations around the TNP apparatus 310. As one example, from the output of a motion sensor, the TNP apparatus 310 can be determined to have been oriented upside-down or used by an individual that may have been walking or positioned within a vehicle or an airplane. As another example, from the output of a temperature sensor, the TNP apparatus 310 can be determined to have been exposed to temperatures outside of a permissible operating temperature range for one or more components of the TNP apparatus 310.

The recorder transceiver 326 can be used to communicate with the device controller 311, the device transceiver 317, or the data processing system 330. The device transceiver 317 can, for example, transmit the sensor data collected by the recorder controller 321 or stored by the recorder memory device 322 via wireless communications (for example, via a communication network like a cellular communications network) or a communication port of the data recorder apparatus 320 or the TNP apparatus 310. The device controller 311, the device transceiver 317, or the data processing system 330 can, in turn, process the sensor data to troubleshoot an error condition of the TNP apparatus 310. The error condition may prevent performance of wound therapy with the TNP apparatus 310 or can be a failure or malfunction of the TNP apparatus 310. The error condition can, for example, be a blockage in a fluid flow path, a low pressure level at a wound, or an expose to a temperature that exceeds a permitted operating temperature for a component like a battery, among other possibilities.

The data recorder apparatus 320 or its components may not communicate with the device controller 311, the device memory device 312, the pressure source 313, the device user interface 314, the device power source 315, the pressure sensor 316, and the device transceiver 317 in some implementations. The data recorder apparatus 320 may accordingly monitor for an error condition at one or more other components of the TNP apparatus 310 without interacting or communicating with the one or more other components.

The TNP apparatus 310 may include additional, alternative, or fewer components than is illustrated in FIG. 3. For example, the data recorder apparatus 320 may not include the recorder user interface 323, so a user may not directly provide inputs to or receive outputs from the data recorder apparatus 320. The data recorder apparatus 320 can instead provide any output of data via the recorder transceiver 326.

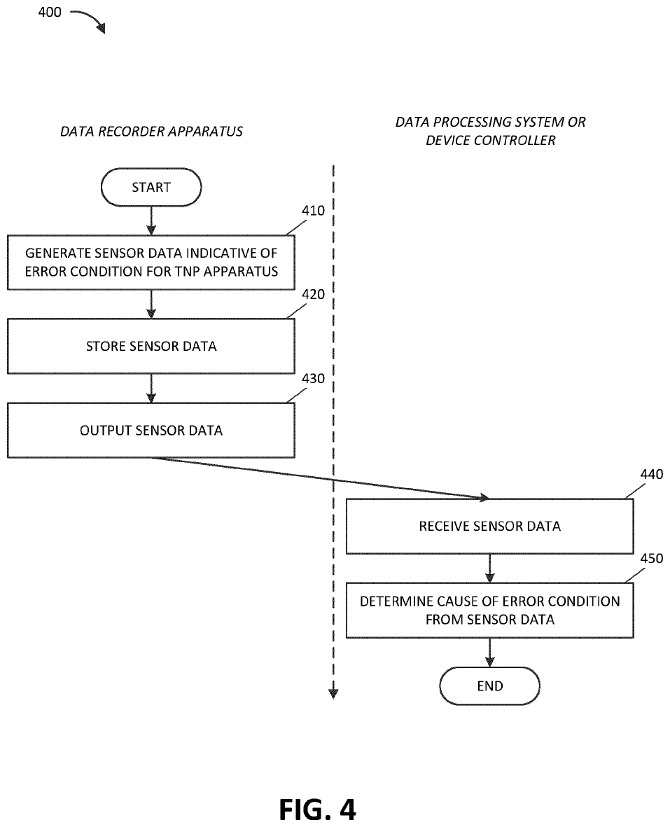

FIG. 4 illustrates a troubleshooting process 400 performable in a negative pressure wound therapy system, such as the negative pressure wound therapy system 300 of FIG. 3. For convenience, the troubleshooting process 400 is described in the context of the negative pressure wound therapy system 300, but may instead be implemented in other systems described herein or by other systems not shown. The troubleshooting process 400 can advantageously, in certain embodiments, enable collection of sensor data with the data recorder apparatus 320 to facilitate successful troubleshooting of an error condition that impacts a functionality of the TNP apparatus 310.

At block 410, the one or more recorder sensors 325 can generate sensor data indicative of an error condition for the TNP apparatus 310. For example, the one or more recorder sensors 325 can include a motion sensor that generates motion data indicative of an improper handling of the TNP apparatus 310. In another example, the one or more recorder sensors 325 can include a temperature sensor that generates temperature data indicative of a temperature of the TNP apparatus 310 which may indicate that the TNP apparatus 310 was exposed to a high temperature environment.

At block 420, the recorder controller 321 can store the sensor data to the recorder memory device 322. At block 430, the recorder transceiver 326 can output the sensor data to the device controller 311 or the data processing system 330. At block 440, the device controller 311 or the data processing system 330 can receive the sensor data from the recorder transceiver 326.

At block 450, the device controller 311 or the data processing system 330 can determine a cause of the error condition from the sensor data. For example, the device controller 311 or the data processing system 330 may determine from motion data that the TNP apparatus 310 was improperly handled and caused a filter of the TNP apparatus 310 to become occluded or a leak or a blockage in the fluid flow path. In another example, the device controller 311 or the data processing system 330 may determine from temperature data that the TNP apparatus 310 was exposed to a high temperature environment and accordingly failed to operate properly (for instance, because a power source of the TNP apparatus 310 failed to charge due to the safety risk of charging the power source in the high temperature conditions).

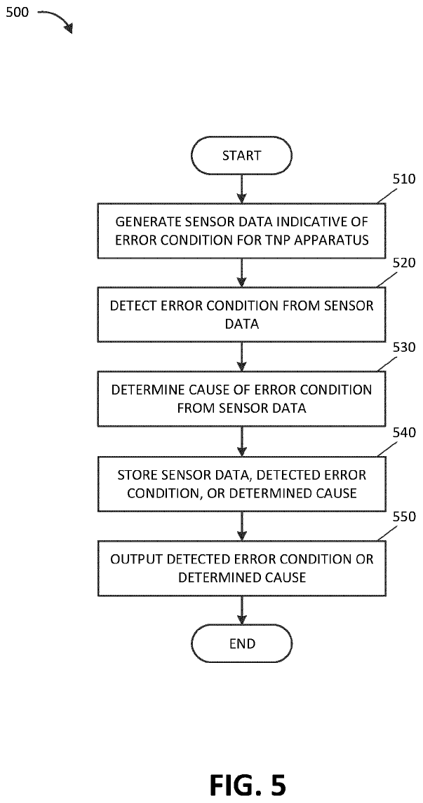

FIG. 5 illustrates a troubleshooting process 500 performable in a negative pressure wound therapy system, such as the negative pressure wound therapy system 300 of FIG. 3. For convenience, the troubleshooting process 500 is described in the context of the negative pressure wound therapy system 300, but may instead be implemented in other systems described herein or by other systems not shown. The troubleshooting process 500 can advantageously, in certain embodiments, enable collection of sensor data with the data recorder apparatus 320, as well as troubleshooting of an error condition that impacts a functionality of the TNP apparatus 310 with the data recorder apparatus 320 from the sensor data.

At block 510 can be similar to or the same as at block 410.

At block 520, the recorder controller 321 can detect an error condition from the sensor data. For example, the recorder controller 321 can analyze motion data to identify motion features (such as an excessive acceleration or an improper orientation of the TNP apparatus 310) indicative of an improper handling of the TNP apparatus 310. As another example, the recorder controller 321 can analyze temperature data to identify temperature features (such as an excessive temperature at the TNP apparatus 310) indicative of a potential failure or disabling of a component of the TNP apparatus 310.

At block 530, the recorder controller 321 can determine a cause of the error condition. For example, the recorder controller 321 can determine from the motion features the specific way in which the TNP apparatus 310 was handled (such as the way in which the TNP apparatus 310 was incorrectly rotated, vibrated, or oriented) that caused the error condition. As another example, the recorder controller 321 can determine from the temperature features how the temperature around the TNP apparatus 310 may have impacted a functionality of the TNP apparatus 310 (for instance, because a power source of the TNP apparatus 310 failed to charge due to the safety risk of charging the power source in a high temperature environment).

At block 540, the recorder controller 321 can store the sensor data, the detected error condition, or the determined cause to the recorder memory device 322. At block 550, the recorder user interface 323 can output the detected error condition or the determined cause for presentation, or the recorder transceiver 326 can output the detected error condition or the determined cause to the device controller 311 or the data processing system 330.

In addition to or instead of outputting the detected error condition or the determined cause, the data recorder can operate differently responsive to the detected error condition or the determined cause, such as by increasing an amount or frequency sensor data collected (which may desirably increase the chances of obtaining sufficient data for troubleshooting the error condition) or turning off to protect the data recorder apparatus from damaging external conditions (such as a fire).

### Terminology and Other Variations

Although some examples herein are described in the context of wound therapy systems, the features (such as of a data recorder) can also be utilized in other systems, such as those systems that involve a device which a user handles and possess but the user has limited understanding of how the device functions or how to troubleshoot the device. The data recorder can serve to collect sensor data that may be useful for troubleshooting the device, such as at a later time or potentially even after an error associated with the device may have been corrected without user intervention. An example of a one such device can be a surgical device where a user may take the surgical device out of a care setting, such as a hospital, and away from easy access a caregiver that may be more knowledgeable about operations or functionality of the surgical device.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. For instance, the various components illustrated in the figures may be implemented as software or firmware on a processor, controller, ASIC, FPGA, or dedicated hardware. Hardware components, such as processors, ASICs, FPGAs, and the like, can include logic circuitry. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. An apparatus for applying pressure to a wound, the apparatus comprising:
a first housing;
a pressure source supported by the first housing, the pressure source being configured to couple via a fluid flow path to a wound dressing positioned on a wound and provide negative pressure to the wound;
a first controller configured to operate the pressure source;
a second housing supported by the first housing;
a sensor within the second housing and configured to generate sensor data usable to troubleshoot an error condition associated with performance of pressure therapy with the first controller; and
a second controller within the second housing and configured to store the sensor data to a memory device, the second controller being different from the first controller.

2. The apparatus of claim 1, wherein the second controller is configured to:
receive device data from the first controller; and
store the device data to the memory device.

3. The apparatus of claim 2, wherein the device data comprises device events or parameter measurements that are associated with operation of the first controller or the pressure source.

4. The apparatus of claim 1, wherein the second controller is configured to add an entry to a log indicating an occurrence of the error condition.

5. The apparatus of claim 4, wherein the second controller is configured to determine a frequency of occurrence of the error condition from the log.

6. The apparatus of claim 1, wherein the second housing is fireproof or shockproof.

7. The apparatus of claim 1, wherein the memory device is non-wipeable or non-resettable.

8. The apparatus of claim 1, wherein the second controller is configured to output the sensor data from the memory device to another device.

9. The apparatus of claim 1, wherein the sensor is a temperature sensor or a motion sensor.

10. The apparatus of claim 1, wherein the first controller and the second controller are powered by different power sources.

11. The apparatus of claim 1, wherein the second controller is configured to:
determine a cause of the error condition from the sensor data; and
output an indication of the cause for presentation on a display.

12. The apparatus of claim 1, wherein the second controller is configured to determine and output a user instruction indicating how to remedy the error condition.

13. The apparatus of claim 1, further comprising a display supported by the second housing, the display comprising electronic ink or one or more light sources.

14. The apparatus of claim 1, wherein the second controller is not configured to operate the pressure source or communicate with the first controller.

15. The apparatus of claim 1, wherein the error condition comprises a blockage in the fluid flow path or a low pressure level at the wound.

16. The apparatus of claim 1, wherein the error condition prevents performance of the pressure therapy with the first controller.

17. A method comprising:
generating, with a sensor, sensor data usable to troubleshoot an error condition associated with operation of a wound therapy device, the sensor being positioned in a recorder housing that is positioned in a device housing of the wound therapy device;
storing the sensor data to a memory device positioned in the recorder housing; and
outputting the sensor data from the memory device to an electronic device.

18. The method of claim 17, further comprising removing the recorder housing from the device housing.

19. The method of claim 17, wherein the sensor comprises a motion sensor, and the sensor data comprises motion data.

20. The method of claim 17, wherein the sensor comprises a temperature sensor, and the sensor data comprises temperature data.

21. (canceled)
